(19) United States
(12) Patent Application Publication
(10) Pub. No.: US 2006/0184144 A1
(43) Pub. Date: Aug. 17, 2006

(54) Fecal specimen collectors

(76) Inventors: Leslie Goulden, Los Altos, CA (US); Joyce Norell, Ben Lomond, CA (US)

Correspondence Address: SHELDON & MAK, INC, 225 SOUTH LAKE AVENUE, 9TH FLOOR, PASADENA, CA, 91101, US

(21) Appl. No.: 11/049,336

(22) Filed: Feb. 1, 2005

(51) Int. Cl. A61M 1/00; A61B 10/00; A61B 5/00

(52) U.S. Cl. 604/317; 600/562; 600/573

(57) ABSTRACT

Methods of collecting a fecal specimen using a fecal specimen collector made from ice, and kits for performing such methods, are provided.

### BACKGROUND

Samples from fecal specimens are used in certain medical diagnostic assays, for example to determine the presence of undigested food particles or parasites. Such samples can also be cultured in order to determine the presence of bacteria, fungi, viruses or protozoa in the fecal material. In addition, fecal samples can be tested chemically in diagnostic assays for the presence of occult blood, bilirubin, and other materials.

Various devices for collecting fecal specimens are known. One such device comprises a receptacle which is suspended over the water line of a toilet by supports attached to the toilet. After defecating into the receptacle, an individual can obtain a sample of the feces in the receptacle, after which the remainder of the fecal specimen is flushed down the toilet. The receptacle and in some cases the supports are then disposed of.

Other fecal specimen collectors comprise a sheet of paper material that floats on the water in a toilet bowl. Feces is deposited on the floating paper material, and a fecal sample can be collected from the fecal specimen supported on the paper. The paper material and remaining fecal matter can then be flushed down the toilet.

Fecal specimen collectors made from paper are readily flushable because the material used for such collectors rapidly disintegrates when wet. This attribute, however, presents a problem for the collection of a fecal sample with such collectors, as the paper's loss of tensile strength shortly after contact with water can result in the collector being punctured by a fecal specimen upon deposition, allowing the specimen to sink below the surface of the water. This can make it difficult to obtain a sample from the specimen, as well as potentially causing it to become contaminated or diluted.

The disposal of used fecal specimen collectors involves either the removal of a collector to a waste bin after use or otherwise flushing a used collector down a toilet. The disposal of collectors in a waste bin has the disadvantage of exposing the disposer to the risk of coming into contact with fecal material remaining in the collector, and in addition contributes to environmental waste. Flushing a fecal specimen collector down a toilet, when possible, runs the risk of blocking drainage pipes due to the bulk of the collector.

### SUMMARY

In one aspect, the present invention provides a method of collecting a fecal specimen by placing a piece of formed ice in a toilet bowl and then providing the fecal specimen on the upper surface of the piece of formed ice. The formed ice has an upper surface for retaining a fecal specimen which is preferably substantially flat, concave, or ridged. In this method, after depositing a fecal specimen on the piece of ice, a sample of feces from the specimen is collected. The fecal sample can then be subjected to a diagnostic assay, such as a fecal occult blood test.

Providing the piece of formed ice in this method generally involves filling a mold with a liquid and then freezing the liquid, thereby forming the piece of formed ice. Preferably, the mold comprises a piece of flexible material, in which case the step of freezing liquid in the mold preferably comprises placing the mold onto a substantially flat surface during the freezing process, thereby forming a substantially flat upper surface in the ice formed in the mold. Such a mold can be attached to a second mold prior to being filled with a liquid, such as via perforations or another type of detachable linker. Molds attached in this way can be provided on a roll for ease of dispensing.

In another aspect, the present invention comprises a kit for performing a diagnostic assay which requires that a fecal sample be obtained. Such a kit comprises a mold for forming a fecal specimen collector comprising formed ice and a fecal sample collector for retaining a fecal sample taken from a fecal specimen. In one embodiment, the fecal sample collector comprises a test device for performing a diagnostic assay, such as a fecal occult blood test, although such a test device can also be supplied in the kit as a separate article. One or more reagents for performing the diagnostic assay can also be provided with the present kits. Such kits further preferably include an applicator stick for removing a fecal sample from a fecal specimen. In addition, a container for retaining the fecal sample collector after a fecal sample has been applied to it can advantageously be included in the present kits. The molds provided with such kits are preferably made from a flexible material, and a plurality of molds is advantageously provided with each kit.

In another aspect, the present invention provides a method of collecting a fecal specimen by placing into a toilet bowl a piece of flexible material which is inflated with air and sealed. A fecal specimen is then provided on the upper surface of the piece of flexible material, which supports the fecal specimen so that a fecal sample can be removed from it. The piece of flexible material is adapted to retain a fecal specimen and to fit within a toilet bowl.

### DRAWINGS

These and other features, aspects and advantages of the present invention will become better understood with regard to the following description, appended claims, and accompanying figures where:

FIG. 1 is a top plan view of a mold for making a fecal specimen collector as described herein.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by their intended use.

### DESCRIPTION

Definitions

As used herein, the following terms have the meanings given below, unless a different meaning is clearly intended by the context in which such term is used.

“Diagnostic assay” refers to a qualitative or quantitative test for determining the presence or quantity of an analyte in a sample for the purpose of diagnosing a disease or medical condition.

“Fecal specimen” refers to a piece of feces discharged from the bowels of a subject, generally a human subject, during a bowel movement. A specimen of feces can comprise some or all of the feces discharged during a particular bowel movement. A “fecal sample” refers to a portion of feces removed from a fecal specimen.

“Formed ice” refers to ice formed outside of a toilet bowl for use as a fecal specimen collector as described herein.

“Ice” refers to a frozen material which exists as a liquid at room temperature (about 22° C.-25° C.). Ice is typically frozen water, i.e. water in the solid state. Ice can contain air or other gas bubbles, solutes, particulates and other entrapped matter in addition to the room temperature liquid, and such solutes and particulates can be either naturally occurring in the liquid or can be added to it prior to freezing.

“Mold” refers to an article which is adapted to retain a liquid and to impart a desired shape to ice formed when such liquid is frozen. When the mold is made from a flexible material, it is understood that the desired shape can in part be imparted by a rigid surface with which the outer surface of the mold is in contact.

“Toilet” as used herein refers to a plumbing fixture for defecation and urination which includes a toilet bowl.

“Toilet bowl” refers to a receptacle having an opening for receiving human wastes (e.g., feces and urine) at an upper end which is provided with an outlet at a lower end for directing such wastes out of the receptacle. Toilet bowls preferably cooperate with a seat for supporting the buttocks of an individual and with a lid for covering the receptacle opening (i.e., Western-style toilet bowls). Toilet bowls can also however be of the variety commonly found in east Asia and elsewhere comprising a receptacle low to the ground having an opening for receiving human wastes and an outlet but lacking a seat (i.e., a squat-type toilet bowl). “Flushing” refers to the process of directing wastes out of a toilet bowl through the outlet by means of a flow of water.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

Fecal Specimen Collectors

1. Fecal Specimen Collectors Made from Formed Ice

In a preferred embodiment, the present fecal specimen collectors comprise a piece of formed ice which is sized and shaped to fit in a toilet bowl. The piece of formed ice further has an upper surface which is adapted to support and retain a fecal specimen which is deposited on the upper surface of such collector. The upper surface of the fecal specimen collector is preferably substantially flat or concave, though a variety of configurations are possible. In a preferred embodiment, the upper surface includes ridges in order to better retain a fecal specimen deposited thereon. While a convex configuration is generally less preferred for the upper surface of a formed ice fecal specimen collector because of the greater likelihood that a fecal specimen will slide off of a convex surface, such collectors can comprise a convex surface of relatively small curvature if such surface does not usually cause a fecal specimen to slide off when the fecal specimen collector is placed in a toilet bowl and a fecal specimen is deposited thereon from approximately the level of the upper rim of a toilet bowl.

The formed ice provides the primary or sole structural support for retaining a fecal specimen deposited thereon in this embodiment. A fecal specimen collector made from formed ice is preferably approximately one-half inch thick, though collectors having a thickness of less than about one-half inch can also be used. A collector made from water and having a thickness (i.e., the distance from the upper surface to a lower surface) of this magnitude has been found to provide sufficient structural strength and buoyancy to support a fecal specimen deposited thereon. It is preferred that formed ice fecal specimen collectors made from water not be much thicker than this, as thicker collectors have been found to crack when placed into contact with room temperature water, such as the water in a toilet. While cracked collectors can still be used if such cracking does not result in the collector becoming broken into different pieces, cracking can lessen the structural strength of a collector and increase the chance that it will break when a fecal specimen is placed on it. Formed ice fecal specimen collectors are also preferably continuous, i.e. do not include openings that communicate with a lower surface of the collector, in order to provide greater structural support and to avoid contact between the upper surface of the collector and water in a toilet bowl.

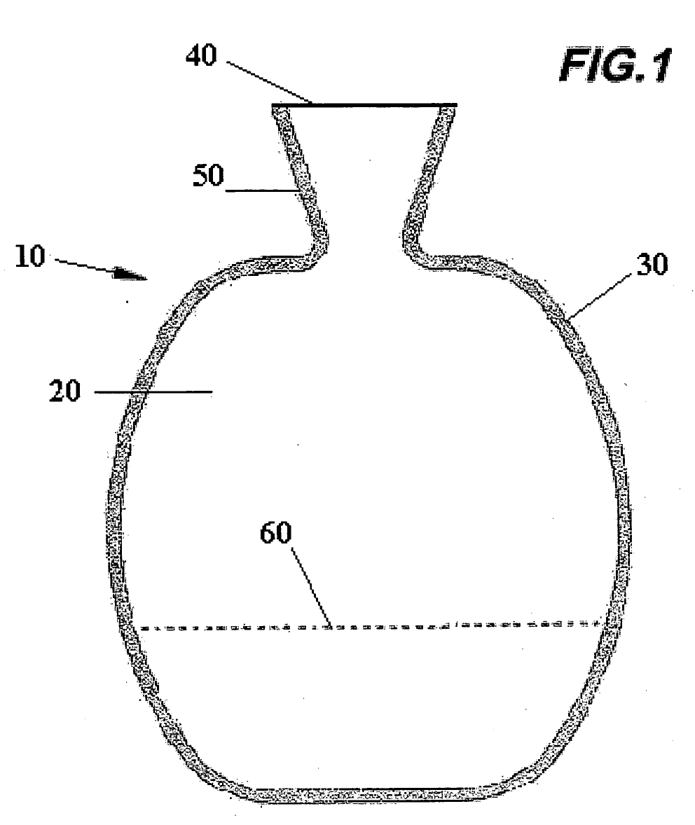

The outer edges of the upper surface of a formed ice fecal specimen collector can comprise any of a variety of shapes, such as an ellipse, circle, square, rectangle, or other regular or irregular shape. Formed ice fecal specimen collectors which are designed to float in the water in a toilet bowl are preferably shaped so as to minimize the amount of space between the edges of the collector and the sides of the toilet bowl, in order to decrease the likelihood of a fecal specimen sliding off the upper surface of the collector and into the water in a toilet bowl. Such collectors should have dimensions approximately equal to or smaller than the dimensions of the surface of the water in the toilet bowl in which they are to be used. The water surface of most Western-style toilet bowls is generally elliptical in shape, with sizes ranging from about 4 inches by 5 inches to about 10 inches by 12 inches (referring to the greatest width and length of the ellipse). In a preferred embodiment, a formed ice fecal specimen collector is in the form of an ellipse approximately 11 inches long and 10 inches wide and is made from a mold 10 as shown in FIG. 1.

Formed ice fecal specimen collectors adapted to float on the surface of water in a toilet bowl are preferably adapted to float such that a suitable area of the upper surface of the collector is above the water line. Preferably, the entire upper surface of the collector floats above the water line. The ability of an object to float is dependent, inter alia, on the density of the fluid (in this case water) in which it rests, as well as on the density of the object (i.e., the fecal specimen collector). The density of pure water ice is 0.92 grams per cubic centimeter, while a cubic centimeter of pure liquid water at 4° C. and atmospheric pressure weighs 1 gram, so that fecal specimen collectors made of pure water ice will float. Most naturally occurring ice has a density less than that of pure ice due to the presence of entrapped air bubbles. A collector adapted to receive a fecal specimen in this embodiment preferably comprises a sufficient mass that the weight of the specimen and collector together divided by the volume of the collector is less than 1 (i.e. so that the collector when combined with the sample has a specific gravity of less than 1, and therefore floats in liquid water).

In other embodiments, the present fecal collectors can be sized so that lower or side portions of such collectors contact the sides of a toilet bowl and support the upper surface of such collectors at or above the level of the water in the toilet bowl. In these embodiments, a fecal specimen is supported on the upper surface of a collector, once deposited, for a sufficient amount of time (i.e., prior to substantial melting) for a fecal sample to be collected from the specimen.

A preferred fecal specimen collector is made solely of formed ice. Such a collector solves the problem of prior fecal specimen collectors of generating waste that must be disposed of, either through removal of the waste from the toilet or through flushing. After use, a collector made solely from formed ice is simply allowed to melt, and the resulting water is flushed down the toilet. The problem of pipe blockage is also solved in this embodiment, as no solid matter (other than the fecal specimen) remains once the fecal specimen collector melts.

The water or other liquid used to make a formed ice fecal specimen collector preferably does not contain additives which would depress the melting point of the ice significantly. The present fecal specimen collectors are preferably used in a setting where the ambient temperature is approximately room temperature, and the collectors are generally used at a temperature above the freezing point of the formed ice. Depressing the melting point of the formed ice in such cases would decrease the amount of time that a collector remains in the solid form and is structurally viable for supporting a fecal specimen at a given temperature, as melting would occur more quickly.

2. Molds

Molds for making a formed ice fecal specimen collector as described herein comprise a cavity for collecting water or other liquid to be frozen. The inner surfaces of the mold cavity in contact with the liquid placed in the mold define, at least in part, the shape of the formed ice fecal specimen collector made with such mold. In one embodiment, the mold can comprise a rigid form or die having one or more cavities for imparting shaped features, in which case the mold is formed from a rigid material such as cardboard or rigid plastic. Rigid molds can be open at the top, or can be closed.

In a preferred embodiment, the mold is made from a flexible material, such as flexible plastic, in which case the mold can preferably be sealed in a water-tight fashion. Such sealing can be accomplished in any of a number of ways, such as by pressure sealing (e.g., with a “zip-loc” mechanism), chemical sealing (e.g., with adhesive), or mechanical sealing (such as through the use of a tie). In one embodiment, liquid is introduced into a mold through a one way valve which retains the liquid inside the mold. Molds which are closed or sealed can further include a fill tube or other conduit for transporting liquid into the mold cavity.

The contours of the present fecal collectors can be imparted entirely by the contours of the inner surface of a mold if the mold is completely filled with water prior to freezing. When a rigid mold is used to produce a fecal collector and the mold is not completely filled (or is open at the top), the upper surface of the water in the mold will freeze and form a flat surface, which advantageously can be the upper surface of the fecal specimen collector. When a flexible mold is used to make the present fecal specimen collectors and the mold is not completely filled, the shape of the fecal specimen collector will be determined in part by a rigid surface on which the mold is placed, as well as by the upper surface of the water in the mold. A flexible mold is advantageously placed on a flat surface during the freezing process in order to impart a flat shape to the upper surface of the collector. Alternatively, such a mold can be placed on a rack or other uneven surface in order to form ridges in the upper surface.

In one embodiment, a plurality of flexible molds are connected, preferably in series. Adjoining molds are preferably connected by detachable linkages to allow the separation of one mold from an adjoining mold. Detachable linkages include, for example, perforated linkages, which can comprise perforations in the material between adjoining molds. Such connected molds can be disposed on a continuous roll, i.e. wrapped around a cylinder. In this embodiment, such a continuous roll can be packaged into a box for dispensing in a similar fashion to the way plastic wrap is dispensed. Molds provided in this form can be advantageously used in a setting where fecal specimens are frequently collected, such as at a hospital.

In an alternative embodiment, a limited number of molds connected via detachable linkages can be advantageously provided with other materials necessary for performing a diagnostic assay which makes use of a fecal sample. One such assay is a fecal occult blood test, for example the HEMOCCULT fecal occult blood test (available from Beckman Coulter, Inc., Fullerton, Calif.). Such tests can require samples from, e.g., three different specimens, in which case kits are provided which comprise at least three molds. In some embodiments, the molds are also packaged together with other diagnostic assay materials for preserving or processing a fecal sample.

FIG. 1 illustrates a preferred embodiment of a flexible mold. In this embodiment, the mold 10 comprises a single interior chamber 20. A liquid such as water is introduced through an opening 40 and a funnel 50 into the interior chamber 20. The mold 10 is elliptically shaped and can be formed from two sheets of polymer material that are heat sealed around the edges 30 of the mold 10. The mold 10 is preferably made from clear polyethylene plastic approximately 0.001″ thick, and is preferably approximately 15 inches long and 11 inches wide.

3. “Balloon” Fecal Specimen Collectors

The flexible molds described above can, in an alternative embodiment, be used as fecal specimen collectors by inflating such molds with air so as to form a “balloon.” This embodiment is advantageous when a freezer is not available to a subject from whom a fecal specimen is needed or when the subject does not wish to wait for water in a mold to freeze so as to form a formed ice fecal specimen collector. In this embodiment, air can be introduced into the flexible molds described above, after which the molds are sealed in an air-tight manner so as to make a balloon. The balloon preferably is filled with air until it is about one-half inch thick. After sealing the funnel or inlet of the mold, the balloon can be floated in a toilet bowl and function as a support for a fecal specimen. After a sample has been collected from the specimen, the balloon can be rinsed off and discarded in a waste container.

In this embodiment, the balloon is preferably not fully inflated. Fecal specimen collectors in this embodiment need only be inflated enough so that they still float when a fecal specimen is deposited on them. The upper surface of a balloon-type collector which is only partially inflated will be depressed in the center when a fecal specimen is deposited thereon, thereby forming a concave upper surface for better retaining the specimen on the collector. A fully inflated fecal specimen collector of this type, by contrast, will generally form a convex configuration, which is not preferred. However, the increased friction between the material used for this embodiment will somewhat mitigate the tendency of a specimen to slide off of such a convex surface and into the water in a toilet bowl. Balloon-type collectors which have a substantially flat upper surface are also possible.

Kits

Molds for the fecal specimen collectors described herein can advantageously be included in kits together with a fecal sample collector. The sample collector can be a container or other article for retaining a fecal sample which is collected using a fecal specimen collector formed using a mold provided in the present kits. Although the user of the present kits can use any clean object to remove a fecal sample from a fecal specimen, such kits usually also include an applicator stick, typically made from wood, for removing a fecal sample from a fecal specimen and transferring or applying the sample or a portion thereof to the fecal sample collector.

The fecal sample collector, in one embodiment, comprises a test device for performing a diagnostic assay with the fecal sample or a portion thereof. For example, the sample collector can comprise guaiac paper for performing a fecal occult blood test. Alternatively, such a test device can be provided in the kit as a separate article, such as the Hemoccult II SENSA elite fecal occult blood test (available from Beckman Coulter, Inc., Fullerton, Calif.), in which case at least a portion of the fecal sample retained by the sample collector is transferred from the sample collector to the test device in order to perform a diagnostic assay. In these embodiments, kits can further include a developing solution or other reagents for performing a diagnostic assay or for preparing the fecal sample to be tested in a diagnostic assay.

In another embodiment, the kit can include a container for retaining the fecal sample collector after a fecal sample has been applied to it. The container can be adapted to be sent, such as via mail or courier, to a laboratory, hospital, or other center where a diagnostic assay can be performed on the fecal sample retained on the fecal sample collector.

Methods of Making Fecal Specimen Collectors

In order to produce a fecal specimen collector with a mold as described above, liquid is placed in the mold, and the mold and liquid together are then placed in an environment at a temperature below the freezing point of the liquid present in the mold. Preferably, the liquid is water, and the freezer is a household freezer. In the case of a rigid mold, the mold can be either open or sealed in a water-tight manner.

When a mold 10 made from a flexible material is used, the mold 10 is preferably sealed in a water-tight manner after being filled with water. In the mold 10 shown in FIG. 1, for example, a knot can be tied in the funnel 50 after water has been introduced into the mold 10 in order to seal it, or a tie or other means of sealing the funnel 50 can be used. Alternatively, the funnel 50 can include a one-way valve which prevents water and/or air from exiting the mold 10 without user intervention.

When a flexible mold is used to form a fecal specimen collector, the flexible mold is preferably placed on a flat or substantially flat surface in a freezer after being filled, so as to form a flat or substantially flat upper surface. A flexible mold can also advantageously be placed on top of a rack in a freezer, preferably one formed from parallel supports with gaps between them. A flexible mold placed on top of such a rack for freezing will tend to conform to that surface such that portions of the mold and the water therein protrude into the gaps between the supports of the rack, thereby forming a ridged surface in the formed ice fecal specimen collector made in this manner. The side of the collector with such ridges can advantageously be used as the upper surface of the collector, as the ridges will tend to inhibit the sliding of a specimen deposited on that surface.

When freezing water or other liquid to make a formed ice fecal specimen collector with a flexible mold, care should be taken to avoid folds or creases in the mold which allow water to freeze in such a way as to trap the mold material between areas of the formed ice product. Should this occur, the formed ice fecal specimen collector will need to be allowed to melt sufficiently so that the trapped mold material can be separated from the collector prior to use.

After a liquid is frozen to make a formed ice fecal specimen collector, the collector is removed from the mold. When a flexible material like polyethylene plastic is used to form the collector, the material is preferably cut and then peeled away from the collector.

Methods of Collecting Fecal Specimens

A fecal specimen is typically collected in order to obtain a sample of such specimen for use in a diagnostic assay. Such a fecal sample is therefore preferably obtained in a way which minimizes contact of the fecal specimen with interfering substances with which it might come into contact after being evacuated from an individual's bowels. In the environment of a toilet bowl, interfering substances can include water, chemicals in the water, and fecal or other material from another individual. Contact with water, for example, can wash away or dissolve trace substances in or on a fecal specimen which are desired to be detected, in particular substances which may be present on the surface of a specimen, such as hemoglobin. Removal of such an analyte by contact with water could lead to a false negative test result. If a fecal sample is contaminated with chemicals present in water in a toilet, the chemicals could interfere with chemical reactions which are performed in a particular diagnostic assay. Material from another individual present in a toilet bowl, if contacted with a fecal specimen, can also cause false test results when the specimen is subjected to a diagnostic assay.

In the present method, it is preferred that a subject from whom a fecal sample is to be collected makes use of a toilet to collect the sample. To collect a fecal specimen, the toilet (preferably free of toilet bowl cleaners and other chemicals) is preferably first flushed. The lid of the toilet, if present, is lifted, and a fecal specimen collector as described herein is placed in the toilet bowl so that the upper surface of the collector is facing upward, i.e. away from the surface of water in the toilet bowl and toward the subject. In order to minimize the potential for contact between a fecal specimen and an interfering substance in the toilet bowl, the fecal specimen collector is preferably placed in the toilet bowl in such a way that the upper surface of the collector does not come into contact with water in the toilet bowl.

After the fecal specimen collector is placed in the toilet bowl, the subject then lowers the seat of the toilet (if it is raised), sits on it and has a bowel movement, thereby providing a fecal specimen on the fecal specimen collector. In embodiments of the invention in which the fecal specimen collector floats on the surface of water in the toilet bowl, the collector should cover enough of the surface area of the water that there is a substantial likelihood that a fecal specimen will be deposited on the collector as a result of a bowel movement by the subject.

Once the fecal specimen is deposited, a fecal sample is collected from the specimen. Typically, fecal samples are collected with a disposable applicator stick, which is used to transfer the sample from the fecal specimen to a fecal sample collector or other support for further analysis. As mentioned above, it is important that the fecal specimen be prevented from contacting the toilet bowl water until after a fecal sample has been taken from it. After a sample has been collected from the fecal specimen, the toilet lid can be closed until the ice has melted of its own accord, after which the toilet can be flushed. Alternatively, the toilet can be flushed in order to melt the ice, for example by flushing three times in succession, which will also thereby dispose of the remaining fecal specimen.

A fecal sample collected in this way can then be subjected to a diagnostic assay, such as a fecal occult blood test, in which case the sample can be contacted with guaiac paper and with a developing solution to detect the presence of hemoglobin in the sample. Other assays can also be performed with such a fecal sample, such as assays to determine the presence of undigested food particles, parasites, bacteria, fungi, viruses, protozoa, or chemical markers such as bilirubin.

### EXAMPLE 1

### Producing a Fecal Specimen Collector

To produce a fecal specimen collector using a polyethylene mold as shown in FIG. 1, the mold 10 was held so that the funnel 50 was upright, and water was then added to the mold 10 through the funnel 50 until the water level in the mold 10 reached the fill line 60 marked on the mold 10. Once the water had been added and a knot tied in the funnel 50, the mold 10 was placed on a flat shelf in the freezer compartment of a household refrigerator. The mold 10 was pulled flat on the shelf to avoid creases in the side of the mold 10 contacting the shelf. After the water was frozen, the resulting ice sheet was slightly rounded on the top and flat on the side that was in contact with the freezer shelf.

The ice sheet in the mold 10 was removed from the polyethylene bag by first cutting off the knot with scissors. The scissors were then inserted through the opening created by cutting off the knot, and the bag was slit from top to bottom. At this point the plastic was peeled back from the ice. The remains of the bag were disposed of in a waste container.

### EXAMPLE 2

### Collecting a Fecal Specimen

A toilet free of toilet bowl cleaners and other chemicals was first flushed. The lid and seat of the toilet were lifted, and the fecal specimen collector made in Example 1 was placed in the toilet bowl so that the substantially flat upper surface of the collector was facing upward. A subject had a bowel movement and deposited a fecal specimen on the collector. Fecal material was collected from the specimen by scraping a small amount of feces from the fecal specimen using a wooden scraper. The fecal specimen was prevented from contacting the toilet bowl water until after the fecal sample had been scraped away.

Although the present invention has been discussed in considerable detail with reference to certain preferred embodiments, other embodiments are possible. The steps disclosed for the present methods are not intended to be limiting nor are they intended to indicate that each step depicted is essential to the method, but instead are exemplary steps only. Recitation of value ranges herein is merely intended to serve as a shorthand method for referring individually to each separate value falling within the range. Unless otherwise indicated herein, each individual value is incorporated into the specification as if it were individually recited herein.

Therefore, the scope of the appended claims should not be limited to the description of preferred embodiments contained in this disclosure. All references cited herein are incorporated by reference to their entirety.

What is claimed is:

1. A method of collecting a fecal specimen, comprising the steps of:
(a) placing a piece of formed ice having an upper surface in a toilet bowl so that a fecal specimen deposited on the upper surface of the piece of formed ice is retained thereon; and
(b) providing the fecal specimen on the upper surface of the piece of formed ice.

2. The method of claim 1, further comprising the step of collecting a fecal sample from the fecal specimen provided on the upper surface of the piece of formed ice.

3. The method of claim 2, further comprising the step of performing a fecal occult blood test on the fecal sample.

4. The method of claim 1, further comprising the step of providing the piece of formed ice prior to step (a).

5. The method of claim 4, wherein providing the piece of formed ice comprises the steps of:
(i) filling a first mold with water;
(ii) freezing the water, thereby forming the piece of formed ice; and
(iii) removing the piece of formed ice from the first mold.

6. The method of claim 5, wherein the first mold comprises a flexible material.

7. The method of claim 6, wherein the first mold is attached to a second mold comprising a flexible material prior to being filled with water.

8. The method of claim 7, wherein the first and second molds are attached by a detachable linkage.

9. The method of claim 7, wherein the first and second molds are disposed on a roll.

10. The method of claim 1, wherein the upper surface of the piece of formed ice has a configuration selected from the group consisting of substantially flat, concave, and ridged.

11. A kit for performing a diagnostic assay, wherein the assay requires that a fecal sample be obtained, comprising:
a mold for forming a fecal specimen collector comprising formed ice; and
a fecal sample collector for retaining the fecal sample.

12. The kit of claim 11, further comprising a test device for performing the diagnostic assay.

13. The kit of claim 11, wherein the fecal sample collector comprises a test device for performing the diagnostic assay.

14. The kit of claim 11, further including a reagent for performing the diagnostic assay.

15. The kit of claim 11, further including an applicator stick for removing the fecal sample from the specimen.

16. The kit of claim 11, further including a container for retaining the fecal sample collector after a fecal sample has been applied to it.

17. The kit of claim 11, wherein the mold comprises a flexible material.

18. The kit of claim 11, further comprising a plurality of molds.

19. The kit of claim 11, wherein the diagnostic assay is a fecal occult blood test.

20. A method of collecting a fecal specimen, comprising the steps of:
(a) placing a piece of flexible material inflated with air in a toilet bowl, the piece of flexible material having an upper surface adapted to retain a fecal specimen; and
(b) providing a fecal specimen on the upper surface of the piece of flexible material.
